(19) United States
(12) Patent Application Publication
(10) Pub. No.: US 2024/0090754 A1
(43) Pub. Date: Mar. 21, 2024

(54) METHOD OF ENDOSCOPICALLY APPROACHING A DUODENUM

(71) Applicants: Daisuke KIKUCHI, Tokyo (JP); OLYMPUS MEDICAL SYSTEMS CORP., Tokyo (JP)

(72) Inventors: Daisuke KIKUCHI, Tokyo (JP); Arimasa SUGIMOTO, Tokyo (JP); Naoya SHIMADA, Tokyo (JP)

(73) Assignees: Daisuke KIKUCHI, Tokyo (JP); OLYMPUS MEDICAL SYSTEMS CORP., Tokyo (JP)

(21) Appl. No.: 17/948,368

(22) Filed: Sep. 20, 2022

(51) Int. Cl. A61B 1/01; A61B 1/00

(52) U.S. Cl. CPC A61B 1/01; A61B 1/00082; A61B 1/00094; A61B 1/00135

(57) ABSTRACT

A method of endoscopically approaching a duodenum includes: inserting an endoscope into a duodenum of a subject through stomach; inserting a venting device into the stomach; and deflating the stomach by removing air from the stomach through the venting device.

### TECHNICAL FIELD

The present invention relates to a method of endoscopically approaching a duodenum.

### BACKGROUND ART

In the related art, endoscopic therapy of body cavities such as stomach, a duodenum, or small intestine has been performed (see PTLs 1 and 2).

PTL 1 discloses a tubular cover for an endoscope. The cover includes a plurality of projecting elements on an outer surface at a distal end portion, and the plurality of projecting elements is adapted to spread in a body cavity and hold an inner wall of the body cavity. By removing air from the inside of the body cavity to deflate the body cavity, a cavity wall enters spaces between the projecting elements and thereby is held at the cover.

PTL 2 discloses a device for performing endoscopic therapy of a trauma under a depressed pressure. The device includes an overtube including a sponge at a distal end, and the sponge is fixed to a cavity wall through suctioning.

### CITATION LIST

### Patent Literature

### PTL 1

U.S. Pat. No. 9,808,142

### PTL 2

U.S. Pat. No. 9,215,964

### Non Patent Literature

### SUMMARY OF INVENTION

### Technical Problem

When endoscopic therapy of a duodenum is performed, there may be following problems. First, forward/backward moving operations and a rotating operation of a proximal portion of the endoscope is unlikely to be transmitted to a distal end. Second, the distal end of the endoscope in the duodenum may unintentionally move back to the inside of the stomach. One of reasons of these problems is that the endoscope is easily movable and deformable inside the stomach.

The present invention has been made in view of the aforementioned circumstances, and an object thereof is to provide a method of endoscopically approaching a duodenum by which it is possible to constantly maintain a path of an endoscope in stomach and to operate a distal end of the endoscope inside the duodenum as desired.

### Solution to Problem

One aspect of the present invention is a method of endoscopically approaching a duodenum, including: inserting an endoscope into the duodenum of a subject through stomach; inserting a venting device into the stomach; and deflating the stomach by removing air from the stomach through the venting device.

### BRIEF DESCRIPTION OF DRAWINGS

FIG. 1A is a diagram for explaining operations of an endoscope and an overtube in a method of endoscopically approaching a duodenum according to an embodiment.

FIG. 1B is a diagram for explaining operations of the endoscope and the overtube in the method of endoscopically approaching a duodenum according to an embodiment.

FIG. 1C is a diagram for explaining operations of the endoscope and the overtube in the method of endoscopically approaching a duodenum according to an embodiment.

FIG. 1D is a diagram for explaining operations of the endoscope and the overtube in the method of endoscopically approaching a duodenum according to an embodiment.

FIG. 1E is a diagram for explaining operations of the endoscope and the overtube in the method of endoscopically approaching a duodenum according to an embodiment.

FIG. 2A is a longitudinal sectional view of the overtube used in the method of endoscopically approaching a duodenum in FIGS. 1A to 1D.

FIG. 2B is a transverse sectional view along the line I-I of the overtube in FIG. 2A.

FIG. 3 is a flowchart of the method of endoscopically approaching a duodenum according to an embodiment.

FIG. 4A is a flowchart of a modification example of the method of endoscopically approaching in FIG. 3.

FIG. 4B is continuation from the flowchart in FIG. 4A.

FIG. 5A is a longitudinal sectional view of a modification example of the overtube in FIGS. 2A and 2B.

FIG. 5B is a transverse sectional view along the line II-II of the overtube in FIG. 5A.

### DESCRIPTION OF EMBODIMENTS

Hereinafter, a method of endoscopically approaching a duodenum according to an embodiment of the present invention will be described with reference to drawings.

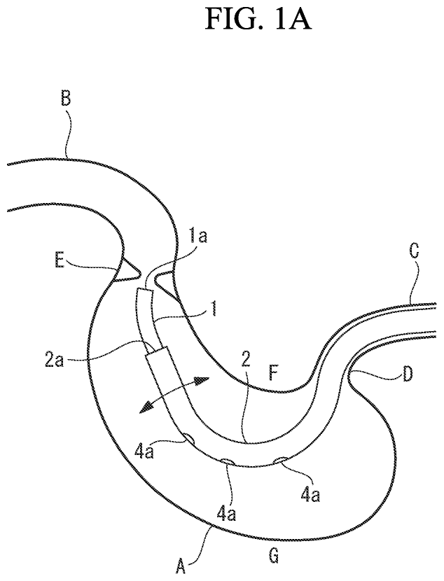

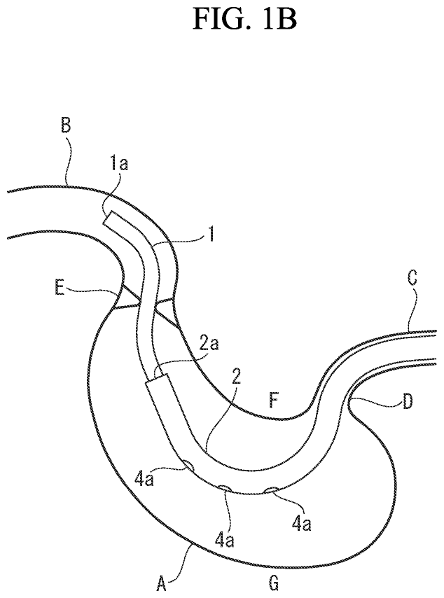

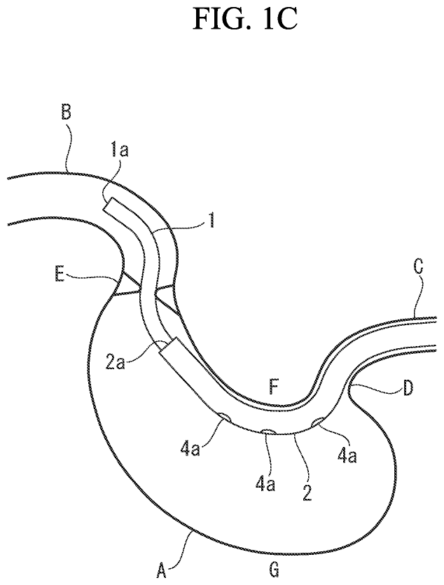

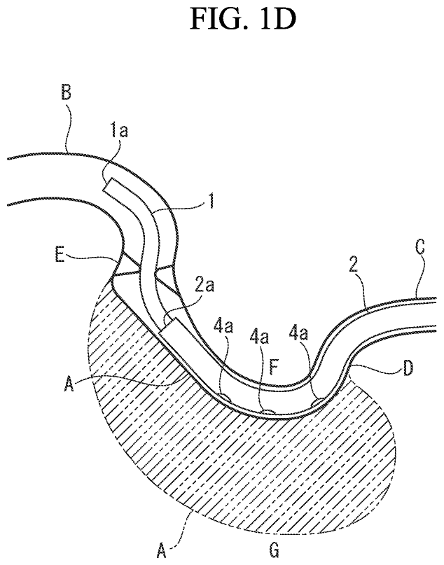

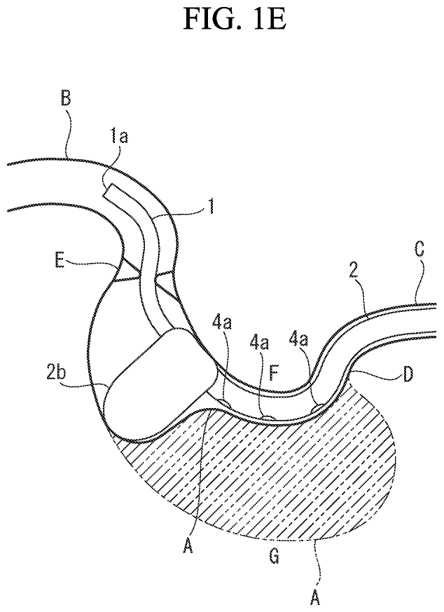

As illustrated in FIGS. 1A to 1D, a method 100 of endoscopically approaching a duodenum according to the present embodiment is a method of inserting an endoscope 1 from a mouth into a duodenum B through a stomach A. The method 100 is realized by using an endoscope system including the endoscope 1 and a venting device 2.

The endoscope 1 is a flexible endoscope for digestive organs.

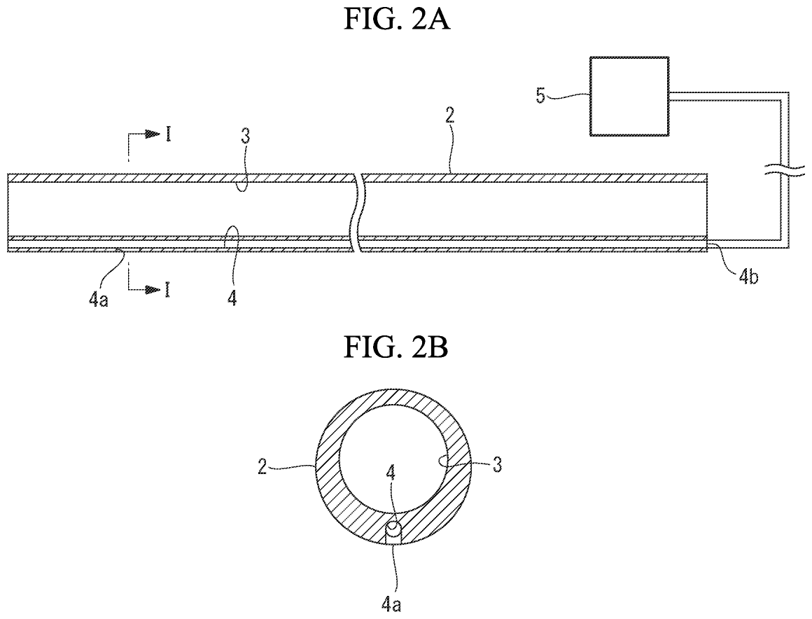

The venting device 2 is a flexible overtube that covers the endoscope 1. As illustrated in FIGS. 2A and 2B, the overtube 2 includes a main lumen 3 into which the endoscope 1 is inserted and a suction lumen 4 for removing air from the stomach A.

The main lumen 3 penetrates through the overtube 2 in the longitudinal direction. The endoscope 1 can move in the longitudinal direction inside the main lumen 3.

The suction lumen 4 includes one or more suction ports 4a and a connection port 4b and extends from the suction ports 4a to the connection port 4b.

The connection port 4b opens in a proximal end surface of the overtube 2 and is fluidically connected to a negative pressure source 5 such as a suction pump.

The one or more suction ports 4a open in an outer circumferential surface of a distal portion of the overtube 2 that is to be disposed inside the stomach A. The overtube 2 may include only one suction port 4a (see FIGS. 2A and 2B) or may include a plurality of suction ports 4a arranged in the longitudinal direction or the circumferential direction (see FIGS. 1A to 1D). The one or more suction ports 4a are fluidically connected to the negative pressure source 5 through the suction lumen 4 and the connection port 4b. Air is suctioned into the suction lumen 4 through the suction ports 4a due to a negative pressure generated by the negative pressure source 5. An opening of the suction lumen 4 in the distal end surface of the overtube 2 may also function as a suction port.

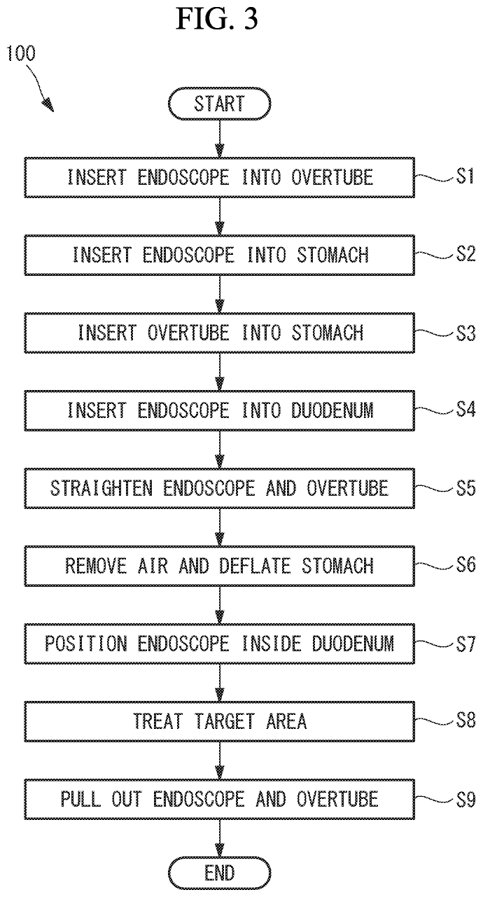

As illustrated in FIG. 3, the method 100 of endoscopically approaching a duodenum includes: Step S1 for mounting the overtube 2 on the endoscope 1; Step S2 for inserting the endoscope 1 into the stomach A of a subject; Step S3 for inserting the overtube 2 into the stomach A; Step S4 for inserting the endoscope 1 into the duodenum B; Step S5 for straightening the endoscope 1 and the overtube 2; Step S6 for removing air inside the stomach A through the overtube 2; Step S7 for positioning a distal end 1a of the endoscope 1 inside the duodenum B; Step S8 for treating a target area; and Step S9 for pulling out the endoscope 1 and the overtube 2 from inside of the body of the subject.

Prior to the insertion of the endoscope 1 into the body of the subject, an operator such as a physician causes the endoscope 1 to pass through the inside of the main lumen 3 of the overtube 2 and causes the endoscope 1 to project from a distal end 2a of the overtube 2 (Step S1).

Next, as illustrated in FIG. 1A, the operator inserts the endoscope 1 extending from the distal end 2a from the mouth into the stomach A of the subject via the esophagus C and the cardia D and disposes the distal end 1a of the endoscope 1 before the pylorus E (Step S2).

Next, the operator inserts the overtube 2 from the mouth into the stomach A along the endoscope 1 and disposes the distal end 2a at an appropriate position inside the stomach A (Step S3). The suction ports 4a are disposed inside the stomach A through Step S3.

Next, as illustrated in FIG. 1B, the operator inserts only the endoscope 1 from the pylorus E into the duodenum B by causing the endoscope 1 to move forward while holding the proximal portion of the overtube 2 disposed outside the body (Step S4).

During Steps S1 to S4, the endoscope 1 and the overtube 2 supported in a cantilever manner at the cardia D are freely movable and deformable inside the stomach A. Therefore, the endoscope 1 and the overtube 2 can be bent and twisted inside the stomach A after Step S4.

Next, as illustrated in FIG. 10, the operator straightens the endoscope 1 and the overtube 2 inside the stomach A by pulling the proximal portions of the endoscope 1 and the overtube 2 disposed outside the body (Step S5). The straightening means causing the bent or twisted endoscope 1 and overtube 2 to approach a straight line shape. As a result of the straightening, the endoscope 1 and the overtube 2 are disposed along the lesser curvature F.

The straightening is performed while preventing the distal end 1a of the endoscope 1 from escaping from the inside of the duodenum B. For example, the distal portion of the endoscope 1 may be hooked at the duodenum B by causing a bending portion of the endoscope 1 to be bent inside the duodenum B. Alternatively, the endoscope 1 may be pulled while causing the endoscope 1 to rotate about the longitudinal axis in a direction in which twist of the endoscope 1 is eliminated. Since the distal end 1a of the endoscope 1 moves forward through the eliminating of the twist, it is possible to straighten the endoscope 1 while preventing the distal end 1a from moving backward in the duodenum B.

Next, the operator removes air inside the stomach A from the suction ports 4a through the suction lumen 4 by causing the negative pressure source 5 connected to the connection port 4b to operate (Step S6). As illustrated in FIG. 1D, the stomach A is deflated by the air being removed, and the stomach wall lateral to the overtube 2 (for example, the stomach wall on the side of the greater curvature G, the anterior side, or the posterior side) approaches the endoscope 1 and the overtube 2. In Step S6, the removing the air is preferably continued until the stomach wall comes into contact with the overtube 2. In FIG. 1D, air is removed until the stomach wall comes into contact with the overtube 2 on the side of the greater curvature. The suctioning of the air performed by the negative pressure source 5 may be stopped after the stomach A is sufficiently deflated.

Next, the operator causes the distal end 1a of the endoscope 1 to move inside the duodenum B and disposes the distal end 1a at a position at which it is possible to treat the target area (Step S7).

Next, the operator treats the target area while observing the target area with the endoscope 1 (Step S8). A treatment tool inserted into the duodenum B through a treatment tool channel of the endoscope 1 may be used for the treatment.

Next, the operator stops the suctioning of air using the negative pressure source 5 and takes the endoscope 1 and the overtube 2 out of the body by pulling out the endoscope 1 and the overtube 2 (Step S9). The endoscope 1 and the overtube 2 may be pulled out at the same time.

In Step S9, the operator may send air to the inside of the stomach A through the overtube 2 or the endoscope 1 prior to the pulling out of the endoscope 1 and the overtube 2. For example, in a case where it is difficult to pull out the overtube 2 due to friction with the stomach wall, the operator may dispose the distal end 1a inside the stomach A by pulling only the endoscope 1 and send air to the inside of the stomach A through a channel of the endoscope 1 until the overtube 2 is released from the stomach wall. Alternatively, the operator may send air to the inside of the stomach A through the suction lumen 4 or the channel of the endoscope 1 until the stomach A is inflated to the original state.

As described above, according to the present embodiment, movement of the endoscope 1 and the overtube 2 inside the stomach A is limited, and the degree of freedom of movement of the endoscope 1 and the overtube 2 inside the stomach A decreases, by deflating the stomach A and reducing the space inside the stomach A. In this manner, since the path of the endoscope 1 inside the stomach A is constantly held, forward/backward movement and rotation of the proximal portion of the endoscope 1 are efficiently transmitted to the distal end 1a, and the distal end 1a inside the duodenum B is prevented from unintentionally returning to the inside of the stomach A. It is thus possible to move the distal end 1a inside the duodenum B as desired by operating the proximal portion.

In particular, in a case where the stomach A is deflated until the stomach wall comes into contact with the overtube 2 after the straightening, the overtube 2 is fixed to the stomach A due to friction with the stomach wall, and as a result, the path of the endoscope 1 inside the stomach A is fixed. It is thus possible to accurately move the distal end 1a inside the duodenum B in accordance with movement of the proximal portion of the endoscope 1.

Also, there is a probability that some of the suction ports 4a are blocked by the stomach wall during Step S6. In a case where the overtube 2 includes a plurality of suction ports 4a, it is possible to reliably remove air inside the stomach A through the other suction port 4a until the space inside the stomach A becomes sufficiently small even after some of the suction ports 4a are blocked.

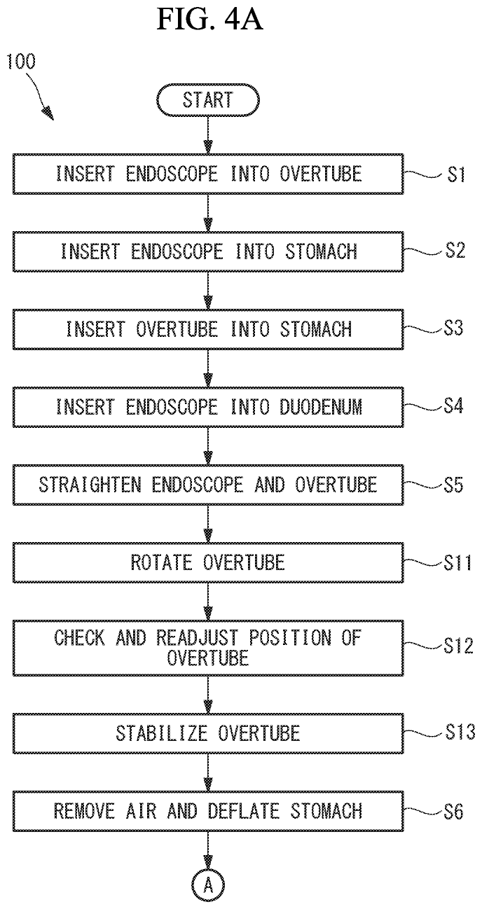

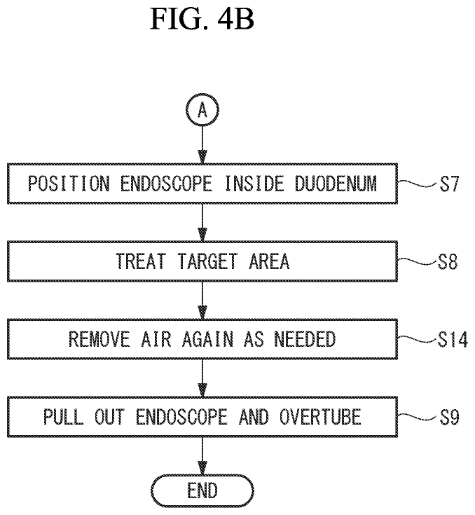

As illustrated in FIGS. 4A and 4B, the method 100 may further include at least one of Steps S11 to S14.

The method 100 may further include Step S11 for causing the overtube 2 to rotate and disposing the suction ports 4a in an appropriate direction after Step S5 and before Step S6.

In a case where the suction ports 4a are disposed on the side of the lesser curvature F, there is a probability that the suction ports 4a are blocked by the stomach wall in an early stage after the suctioning is started. In such a case, it is possible to reliably achieve Step S6 by causing the overtube 2 to rotate and disposing the suction ports 4a on the side of the greater curvature G.

Step S11 is performed by the operator causing the proximal portion of the overtube 2 to rotate. In a case where transmissibility of the rotation of the overtube 2 is low, rotation of the proximal portion is unlikely to be transmitted to the distal portion including the suction ports 4a. Therefore, the overtube 2 preferably includes a reinforcing structure for enhancing transmissibility of rotation about the longitudinal axis. The reinforcing structure is, for example, a blade formed by weaving a fine metal wire or a coil formed by winding a metal wire. The overtube 2 may be a blade tube formed from the blade or a coil tube formed from the coil, or may include the blade or the coil.

The method 100 may further include Step S12 for checking and adjusting the position of the distal end 2a of the overtube 2 inside the stomach A after Step S5.

As a result of performing Step S5, the position of the distal end 2a may be displaced to the proximal side. After Step S5, the operator checks whether or not the position of the distal end 2a is appropriate, and in a case where the position is not appropriate, the operator adjusts the position of the distal end 2a. It is thus possible to ensure operability of the endoscope 1 inside the duodenum B. Step S12 may be performed before or after Step S13, which will be described later.

In Step S12, the checking of the position of the distal end 2a of the overtube 2 may be performed by at least one of the following first, second, and third methods.

The first method is to observe an X-ray perspective image of the stomach A in which the distal end 2a is disposed.

The second method is to observe the distal end 2a using the endoscope 1. In this case, the operator pulls the distal end 1a of the endoscope 1 from the duodenum B back to the stomach A. Subsequently, the operator pulls the endoscope 1 to a position at which the distal end 2a is observed in an endoscope image or bends the bending portion backward and then observes the distal end 2a.

The third method is to check movement of the distal end 1a of the endoscope 1 inside the duodenum B. The operator determines that the position of the distal end 2a is appropriate in a case where the distal end 1a inside the duodenum B moves satisfactorily in response to movement of the proximal portion of the endoscope 1, or determines that the position of the distal end 2a is not appropriate otherwise.

The method 100 may further include Step S13 for stabilizing the overtube 2 after Step S5 and before Step S6. It is possible to further stabilize the path of the endoscope 1 inside the stomach A by performing Step S13. In a case where Step S11 is included, Step S13 is performed after Step S11.

In this case, the overtube 2 includes a stabilization mechanism for stabilizing the overtube 2. The stabilization mechanism may be a shape locking mechanism or a balloon.

The overtube 2 including the shape locking mechanism can transition between a flexible state and a rigid state, for example, by using a shape memory material or by controlling friction between pieces provided at the outer circumference thereof with a tensile force of a wire. It is possible to temporarily fix the shape of the overtube 2 through transition to the rigid state.

The overtube 2 is prevented from moving and deforming inside the stomach A, and the position of the overtube 2 inside the stomach A is stabilized, by fixing the shape of the overtube 2.

As illustrated in FIG. 1E, a balloon 2b is provided at the distal end portion of the overtube 2, and the suction ports 4a are disposed on the proximal side of the balloon 2b. Movement of the balloon 2b inside the stomach A is limited by the stomach wall, and the balloon 2b is held at a constant position, by deflating the stomach A after inflating the balloon 2b. In this manner, the position of the distal end 2a of the overtube 2 is fixed to the stomach A.

After Step S8, the stabilization of the overtube 2 is released by causing the overtube 2 to transition to the flexible state or deflating the balloon 2b.

The method 100 may include Step S14 for removing air again from the stomach A as needed after Step S6.

There is a probability that the stomach A is gradually inflated by air entering the stomach A from the pylorus E or the cardia D after Step S6 and operability of the distal end 1a of the endoscope 1 inside the duodenum B deteriorates. Step S14 is performed, for example, during Step S8. It is possible to recover or maintain the operability of the distal end 1a by performing Step S14.

Step S14 may be performed on the basis of deterioration of the operability of the distal end 1a of the endoscope 1. For example, the operator may perform Step S14 by causing the negative pressure source 5 to operate when the operator recognizes deterioration of the operability.

Step S14 may be performed on the basis of the pressure inside the stomach A. The pressure inside the stomach A is measured by, for example, a pressure sensor inside the suction lumen 4. Step S14 may be automatically performed by the endoscope system. For example, a processor (not illustrated) that controls the negative pressure source 5 may cause suctioning to be started when the pressure exceeds a first threshold value and may stop the suctioning when the pressure drops to below a second threshold value that is lower than the first threshold value. Alternatively, Step S14 may be performed by the operator causing the negative pressure source 5 to be operated when the pressure inside the stomach A drops.

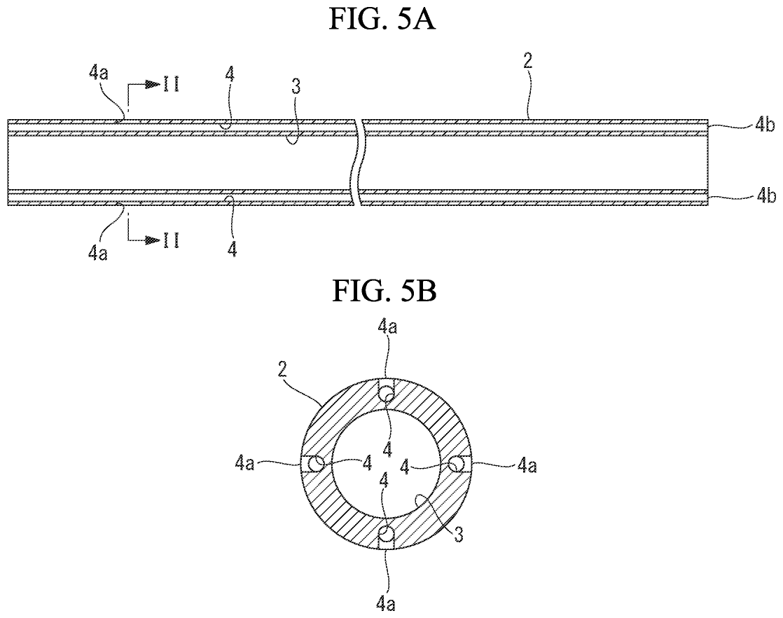

In the aforementioned embodiment, the plurality of suction ports 4a may be disposed at different positions in the circumferential direction of the overtube 2 as illustrated in FIGS. 5A and 5B. With this configuration, any of the suction ports 4a is disposed on the side of the greater curvature G regardless of what orientation about the longitudinal axis the overtube 2 is disposed in, and Step S11 is thus not needed. Also, even in a case where any of the suction ports 4a is blocked with the stomach wall or content of the stomach A, it is possible to continue the removal of air using the other suction ports 4a.

In FIGS. 5A and 5B, the overtube 2 includes a plurality of suction lumens 4 arranged in the surroundings of the main lumen 3, and each suction lumen 4 has the suction port 4a.

The suction lumen 4 may be formed in a helical shape around the main lumen 3, and the suction ports 4a may be aligned in the helical shape. The plurality of suction ports 4a are arranged at different positions in the circumferential direction in this case as well.

In the aforementioned embodiment, the endoscope system may have a function of providing a notification to the operator in a case where the pressure in the suction lumens 4 does not change in a specific period of time after the suctioning is stopped in Step S6.

Typically, the pressure inside the suction lumens 4 gradually raises after the suctioning performed by the negative pressure source 5 is stopped. However, in a case where the suction lumens 4 causes clogging for some reason, the pressure inside the suction lumens 4 does not raise. Therefore, it is possible to cause the operator to recognize abnormality of the suction lumens 4 such as clogging by providing a notification thereto in a case where the pressure does not change after the stopping of the suctioning.

Although the overtube 2 is adapted to be inserted up to the stomach A in the aforementioned embodiment, the overtube 2 may be inserted up to the duodenum B.

In this case, the suction lumens 4 are closed at the distal end surface of the overtube 2, and the suction ports 4a are provided only at a part of the overtube 2 that is to be disposed inside the stomach A.

Although the venting device is the overtube 2 mounted on the endoscope 1 in the aforementioned embodiment, the venting device may be another device instead.

Specifically, the venting device may be an arbitrary device that can be inserted into the stomach A of the subject and can provide an air path between the inside of the stomach A and the outside of the body of the subject. For example, the venting device may be a tube provided with suction lumens that is different from the overtube 2.

Although the embodiment and the modification example of the present invention have been described above in detail with reference to the drawings, specific configurations are not limited to those in the above embodiment and also include changes and the like in design without departing from the gist of the present invention. Also, configurations obtained by appropriately combining components described in the aforementioned embodiment and modification examples can also be employed.

### REFERENCE SIGNS LIST

1 Endoscope
1a Distal end
2 Overtube (venting device)
3 Main lumen
4 Suction lumen
4a Suction port
4b Connection port
5 Negative pressure source
A Stomach
B Duodenum
C Esophagus
D Cardia
E Pylorus
F Lesser curvature
G Greater curvature

1. A method of endoscopically approaching a duodenum, comprising:
inserting an endoscope into the duodenum of a subject through stomach;
inserting a venting device into the stomach; and
deflating the stomach by removing air from the stomach through the venting device.

2. The method of endoscopically approaching a duodenum according to claim 1, further comprising:
straightening the endoscope inside the stomach by pulling a proximal portion of the endoscope after the inserting the endoscope into the duodenum and before the deflating the stomach.

3. The method of endoscopically approaching a duodenum according to claim 2,
wherein the venting device is an overtube into which the endoscope is inserted, and the overtube includes one or more suction ports at a distal portion thereof to be disposed inside the stomach, and
the straightening includes straitening the overtube in the stomach by pulling a proximal portion of the overtube.

4. The method of endoscopically approaching a duodenum according to claim 3, wherein the removing the air is continued until a stomach wall comes into contact with the overtube on a greater curvature side.

5. The method of endoscopically approaching a duodenum according to claim 3, further comprising:
stabilizing the overtube after the straightening and before the deflating the stomach.

6. The method of endoscopically approaching a duodenum according to claim 5, wherein the stabilizing the overtube includes inflating a balloon provided at a distal end portion of the overtube inside the stomach.

7. The method of endoscopically approaching a duodenum according to claim 5, wherein the stabilizing the overtube includes fixing a shape of the overtube by a shape locking mechanism provided in the overtube.

8. The method of endoscopically approaching a duodenum according to claim 3,
wherein the suction port opens in an outer circumferential surface of the overtube, and
the method further comprises:
disposing the suction port on a greater curvature side by causing the overtube to rotate about a longitudinal axis thereof after the straightening and before the deflating the stomach.

9. The method of endoscopically approaching a duodenum according to claim 8, wherein the overtube includes a reinforcing structure of enhancing transmissibility of the rotation about the longitudinal axis.

10. The method of endoscopically approaching a duodenum according to claim 3, wherein the overtube includes a plurality of the suction ports disposed at different positions in a circumferential direction thereof.

11. The method of endoscopically approaching a duodenum according to claim 3, further comprising:
checking and adjusting a position of a distal end of the overtube inside the stomach after the straightening.

12. The method of endoscopically approaching a duodenum according to claim 11,
wherein the checking of the position of the overtube includes at least one of
observing a perspective image of the stomach,
observing the distal end of the overtube with the endoscope, and
checking motion of a distal end of the endoscope in the duodenum.

13. The method of endoscopically approaching a duodenum according to claim 1, further comprising:
removing air from the stomach again on a basis of at least one of operability of a distal end of the endoscope and a pressure inside the stomach after the deflating the stomach.

14. The method of endoscopically approaching a duodenum according to claim 1, further comprising:
treating a target area in the duodenum after the deflating the stomach.

15. The method of endoscopically approaching a duodenum according to claim 14, further comprising:
inflating the stomach by sending air to the stomach through the venting device or the endoscope after the treating.
